(19) United States
(12) Patent Application Publication
(10) Pub. No.: US 2003/0190326 A1
(43) Pub. Date: Oct. 9, 2003

(54) Pharmaceutical composition for immunisation against aids

(76) Inventors: Francois Dalenccon, Lyon (FR); Jean Haensler, Lyon (FR)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/381,951

(22) PCT Filed: Oct. 8, 2001
(86) PCT No.: PCT/FR01/03096

(30) Foreign Application Priority Data
Oct. 6, 2000 (FR) 00/12808

(51) Int. Cl. A61K 39/21; C07H 21/04; A61K 39/00; C07K 1/00; C07K 14/00; C07K 17/00

(52) U.S. Cl. 424/192.1; 424/208.1; 530/350; 536/23.72

(57) ABSTRACT

The invention relates to the field of pharmaceutical compositions for use in immunisation against HIV-related infections, and concerns a pharmaceutical composition comprising at least a HIV antigen and DCchol. Such a composition has proved to be particularly interesting for inducing through the mucous system IgG and IgA specific to the administered antigen. The inventive pharmaceutical composition, in a particular advantageous manner, can be in the form of liposome suspension, or emulsion.

The present invention relates to the field of pharmaceutical compositions for use in immunization against HIV-related infections.

Acquired immunodeficiency syndrome, or AIDS, is a disease against which it would be highly desirable to be able to have a vaccine, and in particular a vaccine for use in prophylaxis. Now, until today, the development of such a vaccine has come up against many problems, including in particular the diverse forms which the HIV virus can take, also the difficulties in identifying the correlates of immunity against HIV, i.e. the immune factors capable of protecting against viruses. Cases of individuals who have been exposed and not infected, and also studies on “non-progressors” give indications, however, and direct research toward the induction of 2 types of immune response: the response by antibodies (humoral response) and the cellular response.

The humoral response that it is desirable to provoke is not only a systemic humoral response, but also a mucosal response since it is desirable to be able to put up an immune barrier against the virus at the site of its entry into the organism.

Research strategies for a vaccine against AIDS are therefore directed toward the search for a vaccine composition capable of inciting the immune system to produce in particular a large amount of IgG and IgA specific for the AIDS virus, in particular in the mucous membranes.

However, this aim comes up against many difficulties, since the HIV antigens liable to be used in a vaccine composition are not always capable of inducing, by themselves, a sufficient immune response, and the formulations known in the prior art for their ability to induce such a response, such as the formulations comprising cholera toxin, sometimes exhibit not insignificant risks of toxicity.

The aim of the present invention is therefore to provide a novel pharmaceutical composition which can be used for immunization, in a prophylactic or therapeutic capacity, against HIV-related infections. Surprisingly, it has been demonstrated that the composition according to the invention makes it possible to induce a large production of IgGs and IgAs specific for an HIV antigen.

To achieve this aim, the present invention provides a pharmaceutical composition comprising at least one HIV antigen and DCchol.

According to one characteristic of the present invention, the HIV antigen is gp160.

According to another characteristic of the present invention, the HIV antigen is p24.

According to another characteristic of the present invention, the HIV antigen is the Tat protein.

According to another characteristic of the present invention, the DCchol is present in the form of a liposomal suspension.

According to another characteristic of the present invention, the DCchol is present in the form of an emulsion.

A subject of the present invention is also a composition comprising at least one HIV antigen and DCchol, for producing a medicinal product for immunization against HIV-related infections.

According to a particular characteristic, the present invention relates to the use of a composition comprising at least one HIV antigen and DCchol, for producing a medicinal product for mucosal immunization against HIV-related infections.

A subject of the present invention is also a method of immunizing a mammal against HIV, according to which at least one HIV antigen and also DCchol are administered to said mammal.

A subject of the invention is also a method of inducing, in the mucosal tissues of a mammal, IgG and IgA antibodies specific for an HIV antigen, according to which a pharmaceutical composition comprising at least one HIV antigen and also DCchol is administered to said mammal.

Other characteristics and advantages of the present invention will become apparent on reading the following description, with reference to the drawings which illustrate the results obtained.

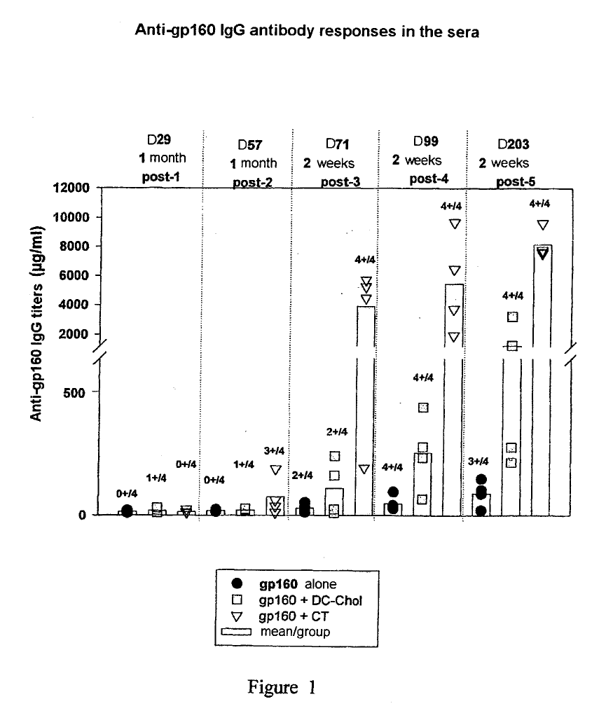

FIGS. 1 to 3 illustrate the results obtained in Example 5.

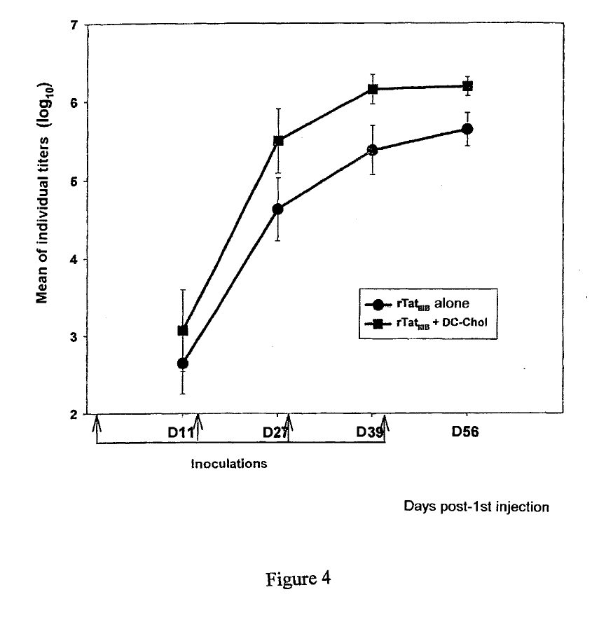

FIG. 4 illustrates the results obtained in Example 6.

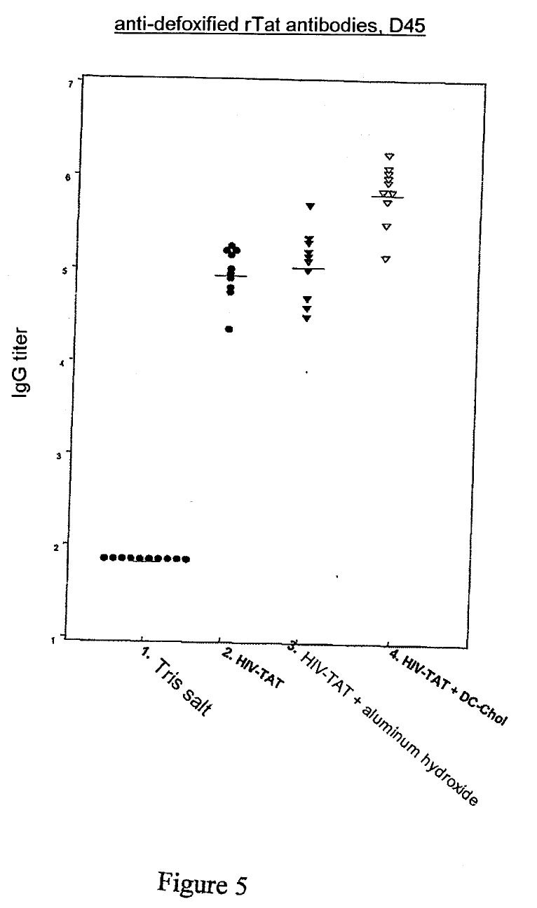

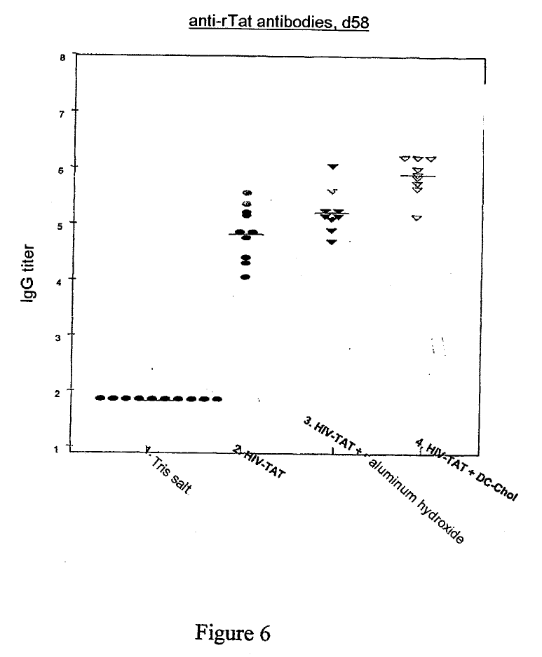

FIGS. 5 and 6 illustrate the results obtained in Example 7.

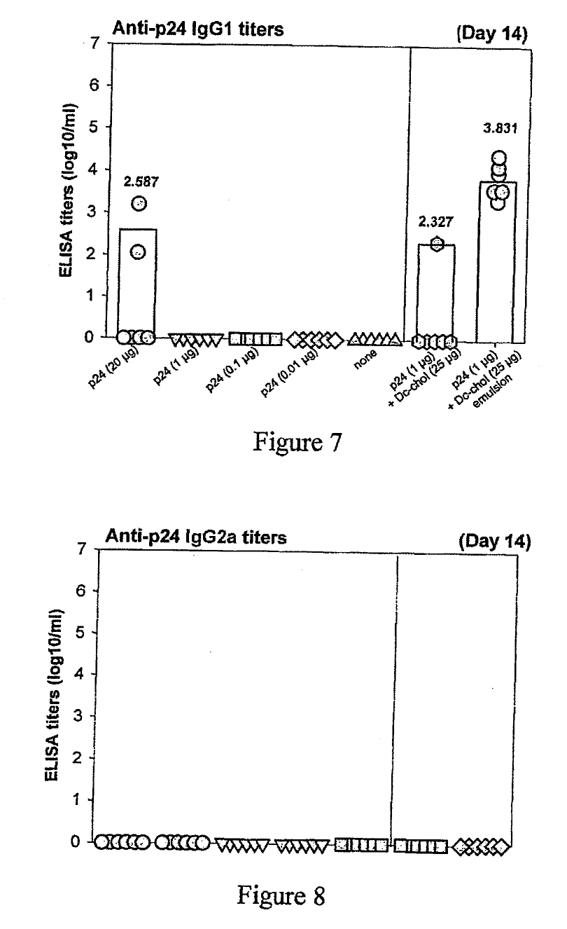

FIGS. 7 to 13 illustrate the results obtained in Example 8.

The term “HIV antigen” is intended to mean any antigen capable of inducing neutralizing antibodies derived from HIV 1 or 2, preferably derived from HIV 1, and including laboratory strains and primary isolates. This term therefore encompasses in particular the surface antigens of the virus, such as glycoprotein 160, gp160, or the proteins derived therefrom, i.e. gp120 and gp41, antigens constituted by internal proteins such as the Gag protein and the proteins deriving therefrom such as p24 and p17, or else the antigens derived from the regulatory proteins such as the Tat protein. The list given above is not limiting. For the purpose of the invention, the term “HIV antigen” is also intended to mean the antigens as defined above which have been modified by chemical or genetic treatment, provided that the treatment applied does not substantially modify the immunological properties of the antigen.

By way of example, mention may be made of the regulatory proteins of HIV detoxified by chemical processes, as described in the following patents or patent applications: U.S. Pat. No. 6,200,575, U.S. 6,132,721, WO 99/33346; the Tat protein detoxified by mutation, as described by Golstein in application WO 95/31999 and Nature Medicine, 1, 960 (1996) and by Loret in application WO 00/61067. Preferably, the Tat protein used in the context of the present invention is a protein lacking its transactivating activity and its immunosuppressor activity.

The suppression of the transactivating activity can be readily controlled using the CAT test as described by G. Tosi et al. in Eur. J. Immuno. (2000) 30, pp. 1120-1126. The suppression of the immunosuppressor activity of the Tat protein can be readily determined using the immunosuppression test as described by Zagury in Proc. Natl. Acad. Sci. USA 1998, 95: 3851-6.

For the purpose of the invention, the term “HIV antigen” is therefore intended to mean the antigen itself, but also the DNA which, after administration by means of a vector such as a canarypox, is capable of being expressed by the organism and therefore of leading to the production in situ of the antigen against which the induction of an immune response is desired. The present invention therefore encompasses, besides the protein antigens defined above, the vectors, preferably viral vectors, expressing these antigens. The viral vectors preferably used in the context of the present invention are the ALVAC and NYVAC vectors which are described in detail in U.S. Pat. Nos. 5,364,773, 5,756,103 and 5,990,091, to which reference may be made for a complete description of these vectors and of the method for obtaining them. The peptide antigen according to the invention may be produced by chemical synthesis or by genetic engineering. The DNA and protein sequences of a large number of HIV antigens are available on the site: http://hiv-web.lanl.gov/ and in the corresponding Los Adamos compendia.

When the antigen is produced by chemical synthesis, the antigen according to the invention can be synthesized in the form of a single sequence, or in the form of several sequences which are then linked to one another. The chemical synthesis can be carried out in solid phase or in solution, these two techniques for synthesis being well known to those skilled in the art. These techniques are described in particular by Atherton and Shepard in “solid phase peptide synthesis (IRL press Oxford, 1989) and by Houbenweyl in “method der organischen chemie” [methods in organic chemistry] edited by E. Wunsch vol, 15-I and II thieme, Stuttgart, 1974, P E Dawson et al. (Science 1994; 266(5186): 776-9); G G Kochendoerfer et al. (Curr Opin Chem Biol 1999; 3(6): 665-71); and P E Dawson et al. (Annu Rev Biochem 2000; 69: 923-60), to which reference may be made for a complete description of the synthesis techniques.

The antigen according to the invention can also be produced by genetic engineering techniques well known to those skilled in the art. These techniques are described in detail in Molecular Cloning: a molecular manual by Maniatis et al., Cold Spring Harbor, 1989. Conventionally, the DNA sequence encoding the polypeptide according to the invention can be produced by the PCR technique, in which the N and C sequences are firstly amplified independently of one another and are then, secondly, paired and amplified once again. The DNA sequence thus obtained is then inserted into an expression vector. The expression vector containing the sequence of interest is then used to transform a host cell which allows expression of the sequence of interest. The polypeptide produced is then isolated from the culture medium by conventional techniques well known to those skilled in the art, such as precipitation with ethanol or with ammonium sulfate, acid extraction, anion/cation exchange chromatography, chromatography on phosphocellulose, hydrophobic interaction chromatography, affinity chromatography, chromatography on hydroxyapatite and chromatography on lectin. Preferably, high performance liquid chromatography (HPLC) is used in the purification.

To carry out the synthesis of the antigen, any expression vector conventionally used to express a recombinant protein can be used in the context of the present invention. This term therefore encompasses both the “live” expression vectors such as viruses and bacteria and the expression vectors of the plasmid type.

Vectors in which the DNA sequence of the antigen according to the invention is under the control of a strong, inducible or noninducible promoter are preferably used. By way of example of a promoter which can be used, mention will be made of the T7 RNA polymerase promoter.

The expression vectors preferably include at least one selectable marker. Such markers include, for example, the dihydrofolate reductase gene or the neomycin resistance gene, for the culturing of eukaryotic cells, and the kanamycin resistance, tetracycline resistance or ampicillin resistance genes, for culturing in E. coli and other bacteria.

By way of an expression vector which can be used in the context of the present invention, mention may be made of the plasmids such as pET28 (novagen) or pBAD (invitrogen) for example.

To promote the expression and the purification of the antigen, the latter can be expressed in a modified form, such as a fusion protein, and can include not only secretion signals, but also additional heterologous functional regions. For example, a region of additional amino acids, in particular charged amino acids, can be added at the N-terminal of the antigen in order to improve stability and persistence in the host cell.

For expression of the antigen, any host cell conventionally used in combination with the expression vectors described above can be used.

By way of nonlimiting example, mention may be made of E. coli cells BL21 (λDE3), HB101, Top 10, CAG 1139, Bacillus, eukaryotic cells such as CHO or Vero.

In the context of the present invention, the following expression vector/cell system will preferably be used: pET(Cer)/BL21LamdaDE3, or BL21lamdaDE3(RIL).

Pharmaceutical compositions suitable for immunization against human immunodeficiency virus type 1 (HIV-1) were prepared, in which the antigen is the gp160 MN/LAI-2 envelope glycoprotein. This antigen contains the gp120 portion of the HIV-1 MN isolate and the gp41 portion of the HIV-1 LAI isolate. The gp41 has had its site of cleavage with gp120, and its transmembrane portion, deleted so as to obtain an uncleaved and essentially secreted glycoprotein. The antigen is produced using the BHK-21 hamster cell line infected with the recombinant vaccinia virus VVTG.9150 derived from the preceding construct VVTG.1163 (M.-P. Kieny, et al., 1988, Protein Eng, 2(3): 219-255), and is then purified by ion exchange chromatography followed by immunoaffinity chromatography.

Pharmaceutical compositions comprising the p24 antigen were also prepared.

This p24 antigen has been described in the following publication: Diagnostic value of HIV-Ag testing and anti-p24 titers in HIV carriers and AIDS patients. A Roumeliotou, E Nestoridou, I Economidou, E Psarra, E Sidiri, E Choremi, G Kallinikos, G Papaevangelou, AIDS 1988 Feb; 2(1): 64.

Pharmaceutical compositions comprising as antigen a detoxified Tat III B protein were also prepared. The Tat protein was detoxified via an alkylation reaction in alkaline medium using iodoacetamide under the following conditions: number of micromoles of iodoacetamide=200×number of micromoles of Tat+number of micromoles of DTT. This detoxified protein and the method for preparing it are described in detail in application WO 99/33346, where it is identified under the term “carboxymethylated Tat”.

The term “DCchol” is intended to mean the product which can be obtained from cholesteryl chloroformate and N,N-dimethylethylenediamine, according to the method described in U.S. Pat. No. 5,283,185 or, preferably, according to the method described in Example 8 of patent application WO 96/40067. It is also possible to use a product obtained by reaction of cholesteryl chloroformate and N,N,N-trimethylethylenediamine.

The vaccine compositions according to the invention can be formulated in various forms, and in particular in the form of emulsions, of liposomes, of liposomes in emulsions, of micelles, etc. Particularly good results have been obtained with emulsions, in particular microfluidized emulsions comprising squalene, and a detergent such as Tween® 80.

According to one characteristic of the invention, the DCchol can also be combined with an immunostimulant oligonucleotide; specifically, it has been noted that, in this case, synergy is obtained between the 2 adjuvants. It is in particular possible to use oligonucleotides such as those described in application WO 96/02555, those described in application EP 0468520, those described in application WO 00/75304, or any other oligonucleotide known for its immunoadjuvant activity.

In particular, the oligonucleotide 3Db(S), the sequence of which is described under SEQ ID NO 15 in application WO 96/02555, can be used.

The pharmaceutical compositions according to the invention are intended to be administered via all the pathways conventionally used in immunization, and in particular parenterally, but also mucosally, in particular orally, nasally, rectally, vaginally. They can be administered according to various protocols, comprising a single step or several steps of administration.

In the case of a protocol in several steps, it is possible for the compositions according to the invention to be administered at each step, or simply at some of them. It is in fact possible for an administration protocol in which a primary immunization step followed by a booster step is envisioned, to have the primary immunization step carried out using a composition different from that of the invention, while the pharmaceutical composition according to the invention will be used only for the booster step.

However, given the qualities of the pharmaceutical compositions according to the invention, it is also possible to use them at each of the steps proposed.

The amount of the antigen in the composition according to the present invention depends on many parameters, as will be understood by those skilled in the art, such as the nature of the antigen, the vector used or the route of administration. A suitable amount is an amount such that a humoral immune response capable of neutralizing primary isolates of HIV is induced after administration of this amount. The amount of protein antigen to be administered is of the order of 10 to 100 micrograms. When the antigen used is a viral vector, the amount of vector to be administered is of the order of 10⁴ to 10⁸ TCID₅₀.

The compositions according to the present invention can be prepared by any conventional method known to those skilled in the art. Conventionally, the antigens according to the invention are mixed with a pharmaceutically acceptable carrier or diluent, such as water or phosphate buffered saline solution. The carrier or diluent will be selected as a function of the pharmaceutical form chosen, of the method and route of administration and also of pharmaceutical practice. The suitable carriers or diluents and also the requirements in terms of pharmaceutical formulation are described in detail in Remington's Pharmaceutical Sciences which represents a reference work in this field.

The following examples illustrate particular embodiments of the present invention.

### EXAMPLE 1

DC-Chol hydrochloride was used (obtained according to the method of preparation described in Example 8 of patent application WO 96/40067), which was suspended at 20 mg/ml in TRIS-NaCl buffer (20 mM TRIS, 150 mM NaCl, pH 6.8). After 8 hours with stirring at 35-40° C. under argon, the suspension was microfluidized using an M-110S microfluidizer from Microfluidics (10 cycles at 500 kPa), in order to generate a homogeneous suspension of DC-Chol, which was filtered through a 0.45 μm Millex filter.

### EXAMPLE 2

Oligonucleotides were prepared using an automatic synthesizer provided by Applied Biosystems, which uses the standard phosphoramidite chemical method and which comprises an oxidation step at each cycle.

This oxidation step was carried out using an iodine/water/tetrahydrofuran/acetonitrile solution so as to obtain a phosphodiester bond, and using a tetraethylthiuram/acetonitrile solution so as to obtain a phosphorothioate bond.

An oligonucleotide 3 Db(S), the sequence of which is reproduced in patent application WO 96/02555 under SEQ ID No. 15, and which comprises phosphorothioate bonds throughout its length, was thus prepared.

An oligonucleotide MGC (S), the sequence of which is reproduced in patent application WO 00/15256 in SEQ ID No. 2, which comprises both phosphodiester bonds and phosphorothioate bonds, was also prepared. The phosphorothioate bonds are located at each end; there are two phosphorothioate bonds positioned 3′ and 5 phosphorothioate bonds positioned 5′. This oligonucleotide contains no CG sequence and is used as a negative control.

### EXAMPLE 3

### gp160+Immunostimulant Oligonucleotide in Mice

Pharmaceutical compositions for immunization against human immunodeficiency virus type 1 (HIV-1) were prepared, in which the antigen is the gp160 MN/LAI-2 envelope glycoprotein. This antigen contains the gp120 portion of the HIV-1 MN isolate and the gp41 portion of the HIV-1 LAI isolate. The gp41 has had its site of cleavage with gp120, and its transmembrane portion, deleted so as to obtain an uncleaved and essentially secreted glycoprotein. The antigen is produced using the BHK-21 hamster cell line infected with the recombinant vaccinia virus VVTG.9150 derived from the preceding construct VVTG.1163 (M.-P. Kieny et al., 1988, Protein Eng, 2(3): 219-255), and is then purified by ion exchange chromatography followed by immunoaffinity chromatography.

The doses for immunization, for 20 μl, corresponded to one of the following formulations:

25 μg of gp160 only,

25 μg of gp160+50 μg of oligonucleotide 3 Db(S) prepared in Example 2,

25 μg of gp160+50 μg of oligonucleotide MGC prepared in Example 2+200 μg of DCchol prepared in Example 1,

25 μg of gp160+50 μg of oligonucleotide 3 Db(S) prepared in Example 2+200 μg of DCchol prepared in Example 1.

4 groups of 6 mice (1 formulation per group) were injected with the prepared doses rectally, under anaesthesia, at a rate of 4 injections each 2 weeks apart (namely D1, D15, D29 and D44).

On D57, serum was taken, the feces were collected and rectal washes were performed in order to titer the following:

titering by ELISA of the anti-gp160 IgGs in the serum,

titering by ELISA of the total IgAs and IgGs and also of the specific anti-gp160 IgAs and IgGs in the rectal washes,

titering by ELISA of the total IgAs and IgGs and also of the specific anti-gp160 IgAs and IgGs in the feces.

The pharmaceutical composition containing the oligonucleotide MGC was considered to be a negative control relative to the oligonucleotide 3 Db(S). Specifically, the oligonucleotide MGC had been shown not to be immunostimulant in preceding experiments. The results obtained are therefore considered to be equivalent to those obtained using DCchol as only adjuvant.

The results obtained are recapitulated in the tables below; only the means per group of mice having received the same composition are indicated.

TABLE 1
Titering of specific IgGs in the serum:

| | Anti-gp160 |
|---|---|
| | IgG in μg/ml |
| 25 μg gp160 | 60.55 |
| 25 μg gp160 + 50 μg 3Db(S) | 46.97 |
| 25 μg gp160 + DCchol 200 μg + 50 μg MGC | 47.85 |
| 25 μg gp160 + DCchol 200 μg + 50 μg 3Db(S) | 645.26 |

These results show the synergy exerted by the two adjuvants for the production of IgG with respect to the gp160 antigen, in a mucosal administration.

TABLE 2
Titering of IgAs and IgGs in the rectal washes:

| | Specific IgA/ | Specific IgG/ |
|---|---|---|
| | total IgA as % | total IgG as % |
| 25 μg gp160 | 0.15 | 3.68 |
| 25 μg gp160 + 50 μg 3Db(S) | 0.91 | 2.46 |
| 25 μg gp160 + DCchol 200 μg + | 0.68 | 1.07 |
| 50 μg MGC | | |
| 25 μg gp160 + DCchol 200 μg + | 1.53 | 12.43 |
| 50 μg 3Db(S) | | |

TABLE 3
Titering of IgAs and IgGs in the feces:

| | Specific IgA/ | Specific IgG/ |
|---|---|---|
| | total IgA × 10⁴ | total IgG as % |
| 25 μg gp160 | 2.44 | 0.00 |
| 25 μg gp160 + 50 μg 3Db(S) | 18.05 | 1.33 |
| 25 μg gp160 + DCchol 200 μg + | 43.38 | 0.00 |
| 50 μg MGC | | |
| 25 μg gp160 + DCchol 200 μg + | 104.79 | 3.03 |
| 50 μg 3Db(S) | | |

These results show the effect of synergy obtained using both DCchol and immunostimulant oligonucleotides, with respect to the local production of specific immunoglobulins A and immunoglobulins G.

This ability to locally stimulate the production of specific IgAs is particularly sought in immunization against HIV-related infections, and confirms the value of the subject of the present invention.

### EXAMPLE 4

### gp160 in Mice

gpl60 as described in Example 3, DCchol as described in Example 1, and cholera toxin CT provided by the company Sigma under the reference #C-8052 are available for use.

Using these products, immunizing compositions comprising the gp160 alone, or gp160 and DCchol, or gp160 and CT, are prepared.

The immunizing compositions are administered nasally to female BALB/c By J Ico mice. The intranasal immunizations are given 4 times, in a proportion of 2×5 μl in each nostril (i.e. 2×5 μl in the morning, and 2×5 μl in the afternoon).

The amounts administered per dose are as follows: 25 μg of gp160, 200 μg of DCchol and 5 μg of CT.

The immunizations took place on days 1, 13, 30 and 59.

The blood samples were taken from the retro-orbital sinus on days 42 and 75, i.e. respectively after 3 and 4 immunizations.

The vaginal secretions were taken after the 3rd and after the 4th immunizations.

The rectal secretions were taken after the mice were sacrificed.

The nasal secretions were taken at the end of the test via a nasopharyngeal wash.

The total IgAs and IgGs and the gp160-specific IgAs and IgGs were assayed by ELISA in the sera, and the nasal, vaginal and rectal secretions.

In summary, NUNC Maxisorp F96 plates were coated with anti-mouse IgA or anti-mouse IgG antibodies or with gp160 MN/LAI-2 and incubated with serial dilutions of serum or of secretion. The total and specific IgAs or IgGs were revealed with an anti-mouse IgA or anti-mouse IgG peroxidase conjugate after addition of OPD (O-phenylenediamine dihydrochloride).

For each secretion, the anti-gp160 IgA or IgG titers were divided by the total IgA or IgG titers (ratios called normalized specific activities, NSA) so as to be able to compare the samples with one another.

The results relating to the serum antibodies obtained are given in Table 4 below, in which it is seen that DCchol is a good adjuvant without, however, reaching the activity of CT, but the CT led to the observation of signs of toxicity.

TABLE 4

| Immunization | Anti-gp160 IgG | Anti-gp160 IgG |
|---|---|---|
| composition | (μg/ml) at D41 after | (μg/ml) at D79 after |
| received | the 3rd immunization | the 4th immunization |
| gp160 (25 μg) | 4.694 | 20.545 |
| gp160 (25 μg) + | 128.698 | 172.022 |
| DCchol (200 μg) | | |
| gp160 (25 μg) + | 325.091 | 430.806 |
| CT (5 μg) | | |

The results relating to the antibodies titered in the nasopharyngeal samples are given in Table 5 below. Only the means of the results obtained after 4 immunizations are indicated. Analysis of the normalized specific activities (NSA) shows a marked adjuvant effect of DCchol on the IgA responses, despite the great individual variabilities of the ratios. Compared to gp160 alone, the NSAs were multiplied by a factor of 12 on average. Thus, in a certain number of mice, a majority of the IgAs harvested from nasopharyngeal washes proved to be specific for the antigen administered.

On the other hand, the adjuvants according to the prior art, CT, did not make it possible to increase the local IgA responses, despite its strong positive effect at the serum level.

TABLE 5

| Immunization | Anti-gp IgA/total | Anti-gp IgG/IgG |
|---|---|---|
| composition received | IgA ratio (%) | ratio (%) |
| gp160 MN/LAI-2 (25 μg) | 4.76 | 0.62 |
| gp160 + DCchol (200 μg) | 55.05 | 8.23 |
| gp160 + CT (5 μg) | 7.28 | 7.81 |

The results relating to the responses measured in the vaginal samples are given in Table 6A (for the IgGs) and 6B (for the IgAs) below, in which only the means of the results are given. As for the nasopharyngeal samples, a good response in terms of the IgGs and the IgAs, obtained with the compositions according to the invention, is noted here, even though the IgA NSAs are lower than previously, the IgG NSAs themselves being of the same order.

The results show that, by virtue of a mucosal administration, it is possible to induce, using immunization compositions according to the invention, a response in another mucosal compartment very distant from the site of immunization.

TABLE 6A

| Immunization | Anti-gp IgG/total | Anti-gp IgG/total |
|---|---|---|
| composition | IgG ratio (%) after | IgG ratio (%) after |
| received | the 3rd immunization | the 4th immunization |
| gp160 (25 μg) | 0 | 0.62 |
| gp160 (25 μg) + | 2.31 | 6.93 |
| DCchol (200 μg) | | |
| gp160 (25 μg) + | 4.53 | 8.26 |
| CT (5 μg) | | |

TABLE 6B

| Immunization | Anti-gp IgA/total | Anti-gp IgA/total |
|---|---|---|
| composition | IgA ratio (%) after | IgA ratio (%) after |
| received | the 3rd immunization | the 4th immunization |
| gp160 (25 μg) | 0 | 0.95 |
| gp160 (25 μg) + | 7.00 | 22.32 |
| DCchol (200 μg) | | |
| gp160 (25 μg) + | 1.82 | 6.75 |
| CT (5 μg) | | |

The results relating to the rectal samples are given in Table 7 below, in which it is seen that the IgA responses are relatively weak, but in which, on the other hand the effectiveness of the immunization compositions according to the invention with regard to the IgGs produced is noted.

TABLE 7

| Immunization | Anti-gp IgA/total | Anti-gp IgG/total |
|---|---|---|
| composition received | IgA ratio (%) | IgG ratio (%) |
| gp160 (25 μg) | 0 | 0.89 |
| gp160 + DCchol (200 μg) | 0.87 | 9.02 |
| gp160 (25 μg) + CT (5 μg) | 0.39 | 8.91 |

### Example 5

### gp160 in Macaques

In the same way as the previous example, gp160, DCchol and cholera toxin CT are available for use and, using these products, immunizing compositions comprising either gp160 alone, or gp160 and DCchol, or gp160 and CT, are prepared.

The intention is to evaluate the effectiveness of these compositions in primates, in an entirely mucosal administration protocol, in particular on the response induced at the mucosal level, but also in the serum.

For this, 12 female rhesus monkeys are divided into 3 groups, and are simultaneously immunized nasally, vaginally and rectally, 5 times (i.e. on days 1, 29, 57, 85 and 190) using the following immunization compositions:

gp160 alone in a proportion of 50 μg per route and per animal,

gp160 and DCchol in a proportion of 50 μg of gp160 and 400 μg of DCchol per route and per animal,

gp160 and CT in a proportion of 50 μg of gp160 and 50 μg of CT per route and per animal.

The volumes administered are as follows:

nasally: 100 μl in each nostril,

vaginally: 200 μl

rectally: 1000 μl.

The samples are taken on the following dates:

sera: D-11/-4, 1, 29, 57, 71, 99, 184 and 203

vaginal secretions: D-11, 71/78, 99/106, 184 and 203/212

nasal secretions: D-11, 71, 99, 184 and 203.

The vaginal samples were taken in duplicate, with a 7 or 9 day interval, in order to avoid possible contamination by menses.

The IgGs and IgAs were titered by ELISA.

In order to be rid of the dilution factor introduced into the mucosal secretions when samples were taken by washing, the anti-gp160 IgA and IgG titers were normalized, respectively, with the total IgA and IgG titers (ratios called normalized specific activities).

The results relating to the anti-gp160 IgG responses measured in the sera are indicated in FIG. 1, in which the effectiveness of the immunization compositions according to the invention, in particular after the 4th and 5th immunizations, is clearly seen.

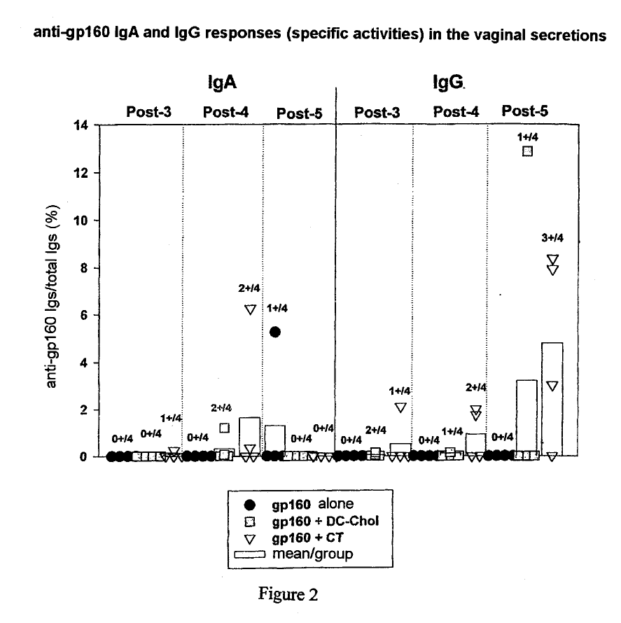

The results relating to the responses obtained in the vaginal secretions are represented in FIG. 2, in which the improvement in the immune response induced using the immunization compositions according to the invention, compared to the responses obtained when gp160 alone is administered can be seen.

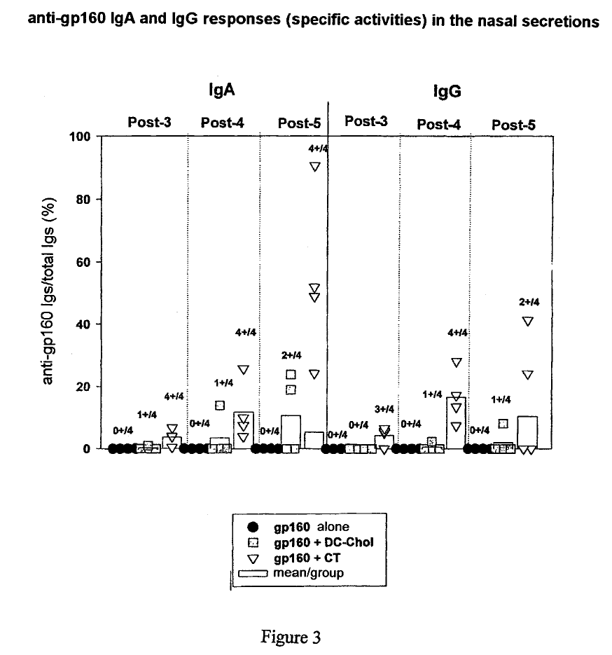

The results relating to the responses obtained in the nasal secretions are represented in FIG. 3, in which an improvement in the responses obtained with the immunization compositions according to the invention, compared to the responses obtained with the antigen alone, is also noted.

### EXAMPLE 6

### Tat Protein in Guinea Pigs

Carboxymethylated Tat protein, obtained by expression in E. coli and purification by various chromatography steps, then chemical inactivation, as described in application WO 99/33346, is available for use.

Immunizing compositions comprising either Tat protein alone in PBS buffer or Tat protein in the presence of DCchol, in a proportion of 500 μg of DCchol per dose, are prepared. The 1 ml immunizing doses comprise 50 μg of Tat protein.

Dunkin-Hartley albino guinea pigs are then used, and are divided into 2 groups of 5 guinea pigs, which are immunized intramuscularly in the thigh, in a proportion of one 0.5 ml injection in each thigh.

The injections are given on days 1, 14, 29 and 43.

The serum samples were taken on days 11, 27, 39 and 56.

The antibodies produced are assayed by the ELISA method.

The results obtained are given in FIG. 4, in which it is clearly seen that the amount of anti-Tat antibodies produced is clearly greater with a pharmaceutical composition according to the invention than with a composition comprising only the antigen alone.

### EXAMPLE 7

### Tat Protein in Mice

Carboxymethylated Tat protein, obtained by expression in E. coli and purification by various chromatography steps, then chemical inactivation, as described in application WO 99/33346, is available for use.

Immunizing compositions comprising the following are prepared:

Tat protein in a proportion 200 μg/ml in TRIS/NaCl buffer

Tat protein in a proportion of 200 g/ml and aluminum hydroxide in TRIS/NaCl buffer,

Tat protein in a proportion of 200 μg/ml and DCchol obtained in Example 1 in TRIS/NaCl buffer.

Sprague Dawley rats are available for use, and are injected with the immunizing preparations, intramuscularly, on days 1, 8, 15, 29 and 43.

At each administration, 2 injections of 0.25 ml are given per animal.

Blood samples are taken on days 45 and 58, in order to titer, by ELISA, the anti-detoxified recombinant Tat IgG antibodies.

The results obtained, expressed as log IgG, are represented in FIG. 5 for the results of D45 and in FIG. 6 for the results of D58.

These results illustrate the effectiveness of the pharmaceutical compositions according to the invention compared to immunization compositions comprising only the Tat antigen.

### EXAMPLE 8

### p24 in Mice

The p24 protein, which is an antigen which was described in the following publication: Diagnostic value of HIV-Ag testing and anti-p24 titers in HIV carriers and AIDS patients. A Roumeliotou, E Nestoridou, I Economidou, E Psarra, E Sidiri, E Choremi, G Kallinikos, G Papaevangelou, AIDS February 1988; 2(1):64, is available for use.

This antigen is present at a concentration of 1 mg/ml in a solution containing 20 mM sodium phosphate, 50 mM NaCl, at pH 7.5.

A solution of TRIS/NaCl containing 20 mM of TRIS and 150 mM of NaCl, at pH 8, and also DCchol obtained according to Example 1, are also available for use.

Immunizing compositions having the following composition are thus prepared:

Group 1: p24 (20 μg/dose)

Group 2: p24 (1 μg/dose)

Group 3: p24 (0.1 μg/dose)

Group 4: p24 (0.01 μg/dose)

Group 5: control consisting only of a saline solution

Group 6: p24 (1 μg/dose)+DCchol (25 μg/dose) in the form of a liposomal suspension

Group 7: p24 (1 μg/dose)+DCchol (25 μg/dose) in squalene/Tween® 80 emulsion in which the amount of squalene is 5 mg/dose and the amount of Tween® 80 is 600 μg/dose. The emulsion is obtained by mixing 1 g of squalene in 20 ml of TRIS/NaCl containing Tween® 80 and DCchol, homogenized with an ultra-turrax for 1 min at 13 500 rpm, and then microfluidized before being filtered. The drops of the emulsion thus obtained have a mean size of around 150 nm.

The various immunizing compositions are administered to BALB/c mice divided into groups of 6, each group receiving a different composition.

Each of the mice receives a dose of 200 μl on D1 and on D21, subcutaneously.

Samples are taken on D14 and on D35 in order to titer the serum antibodies.

On D37, the mice are sacrificed.

Assays for proliferation in response to recombinant p24 protein (5 μg/ml) at 5 days are carried out.

Assays relating to the production of IL5 and of γIFN in the supernatants of spleen cells which are stimulated, or not stimulated, for 5 days in vitro with 10 mg/ml of recombinant p24 are also carried out.

The results obtained are illustrated in FIGS. 7 to 13.

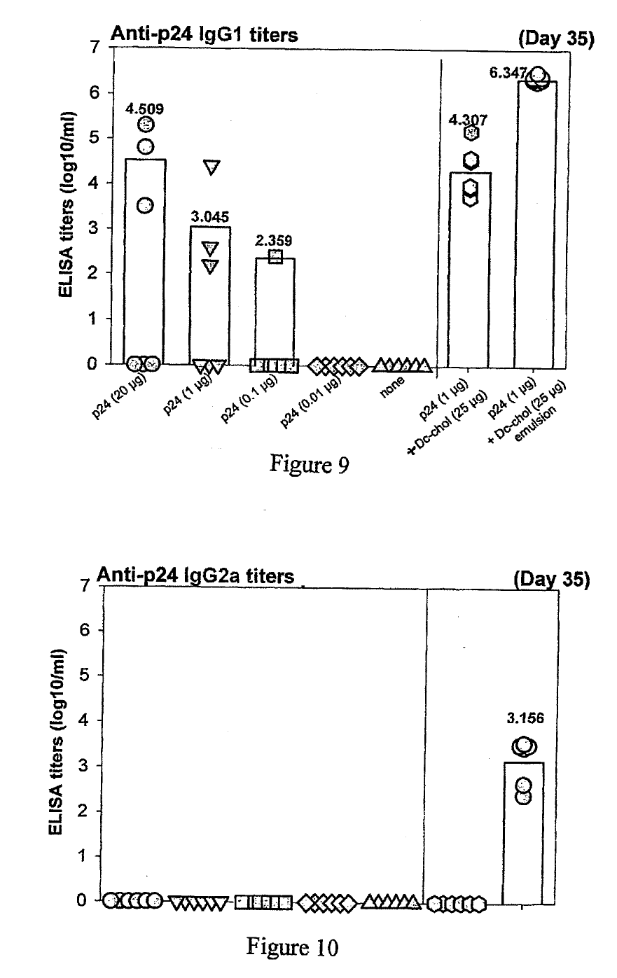

These results show that the IgG1 antibody titers at D14 and at D35 (FIGS. 7 and 9) are better with the pharmaceutical compositions according to the invention than with immunization compositions comprising only the antigen, at the same concentration of antigen, and that, at D35, one of the compositions according to the invention is also capable of inducing the production of IgG2a antibodies (FIG. 10), which is representative of the induction of a TH1-type response.

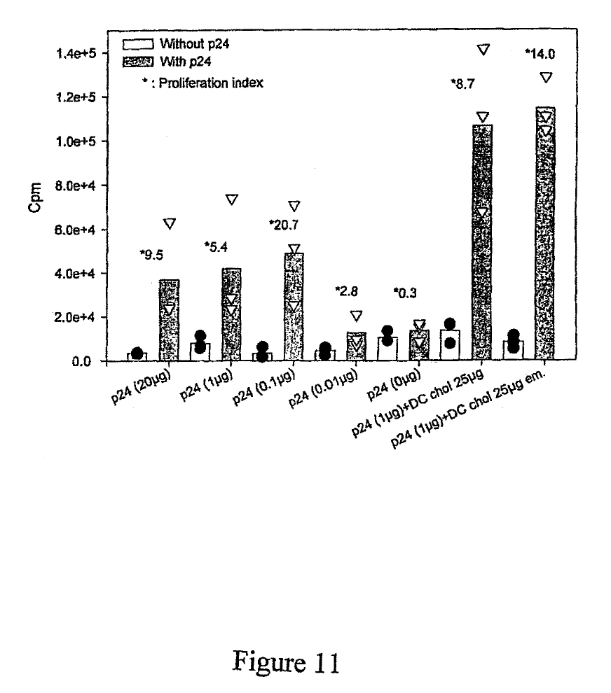

In addition, the immunization compositions according to the invention lead to a higher proliferation index than the compositions comprising only the antigen (FIG. 11).

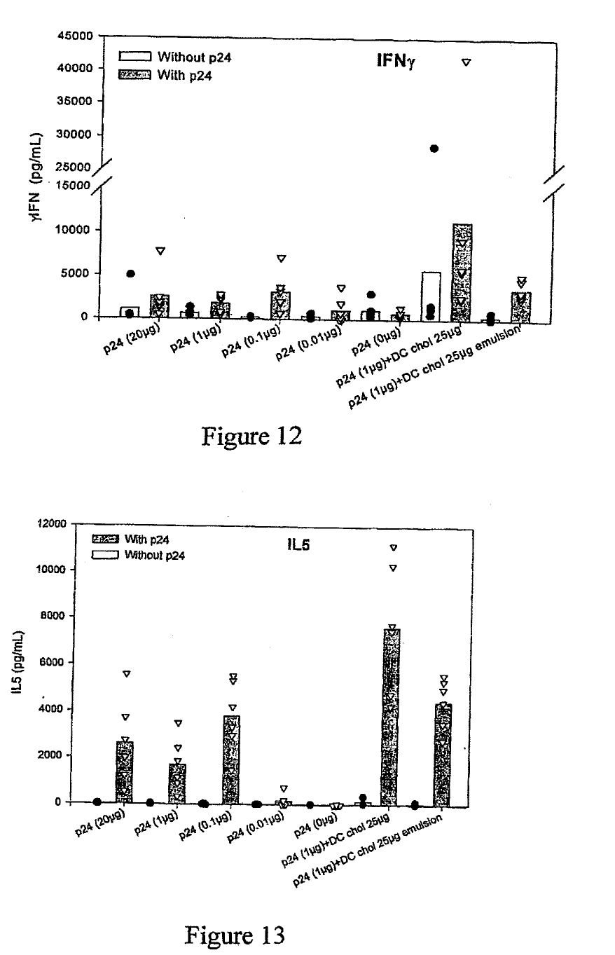

The results of the assays for the cytokines γIFN and IL5 (FIGS. 12 and 13) show that, when an immunization composition according to the invention is used, the amounts of cytokines produced are also increased.

1. A pharmaceutical composition, characterized in that it comprises at least one HIV antigen and DCchol.

2. The pharmaceutical composition as claimed in claim 1, characterized in that the HIV antigen is gp160.

3. The pharmaceutical composition as claimed in claim 1, characterized in that the HIV antigen is p24.

4. The pharmaceutical composition as claimed in claim 1, characterized in that the HIV antigen is the Tat protein.

5. The pharmaceutical composition as claimed in one of the preceding claims, characterized in that the DCchol is present in the form of a liposomal suspension.

6. The pharmaceutical composition as claimed in one of the preceding claims, characterized in that the DCchol is present in the form of an emulsion.

7. The use of a composition comprising at least one HIV antigen and DCchol, for producing a medicinal product for immunization against HIV-related infections.

8. The use as claimed in the preceding claim, characterized in that the immunization is carried out mucosally.

9. The use as claimed in either of claims 7 and 8, characterized in that the medicinal product is intended for the induction, in the mucosal tissues of a mammal, of IgG or IgA antibodies specific for an HIV antigen.

10. A method of immunizing a mammal against HIV, according to which at least one HIV antigen and also DCchol are administered to said mammal.

11. A method of inducing, in the mucosal tissues of a mammal, IgG or IgA antibodies specific for an HIV antigen, according to which a pharmaceutical composition comprising at least one HIV antigen and DCchol is administered to said mammal.
